(19) United States
(12) Patent Application Publication
(10) Pub. No.: US 2010/0010548 A1
(43) Pub. Date: Jan. 14, 2010

(54) Instruments and Method of Use for Minimally Invasive Spine Surgery in Interspine Space Through Only One Side

(76) Inventor: Elias Humberto Hermida Ochoa, Cuauhtemoc (MX)

Correspondence Address: MILLER, MATTHIAS & HULL, ONE NORTH FRANKLIN STREET, SUITE 2350, CHICAGO, IL, 60606, US

(21) Appl. No.: 12/171,859

(22) Filed: Jul. 11, 2008

(51) Int. Cl. A61F 5/00; A61B 17/70

(52) U.S. Cl. 606/86 A; 606/276; 606/237

(57) ABSTRACT

The present disclosure provides a save and single method, as well as a new instrumentation to improve the technique of the minimally invasive spine surgery in only one side with the use of hooks and a new interspinal device of improved stabilization which is placed to decrease the pressure on the affected disk, fastening the intervertebral space and achieving the segmentary stability.

### FIELD OF THE DISCLOSURE

The present disclosure relates to instrumentation and a method associated for the minimally invasive insertion of an implant in spine surgery; particularly, for the insertion of a spinal implant improved through only one side to enhance stabilization of adjacent vertebral bodies.

### BACKGROUND OF THE DISCLOSURE

In minimally invasive surgical procedures of spine, it is important to minimize trauma to patients and damage to tissue to enhance recovery, since having a major tissue cutting results in a more painful post-operative process, greater blood loss, longer hospital staying and recovery. A way to achieve this is by minimizing the sizes of the incision for the surgical procedure and minimizing the tissue cutting to access the spine as well as the use of interspinal devices for stabilization in adjacent spinal vertebra. There are several techniques to expand a small surgical incision and provide access to minimally surgical zones. A common procedure is inserting a graft, a pin or a screw in a hole formed and prepared in the bone or the fine tissues to enhance operation and curative state. See, for example: European Patent No. EP880938A1 “Instrumentation for implant insertion”; U.S. Application No. US20050228380A1 “Instruments and methods for minimally invasive spine surgery”; U.S. Application No. US2008045957 “Spinal Stabilization Systems and Methods Using Minimally Invasive Surgical Procedures”; U.S. Application No. US2008021285 “Surgical retractor for use with minimally invasive spinal stabilization systems and methods of minimally invasive surgery”; U.S. Pat. No. 5,891,147 “Minimally invasive spinal surgical methods & instruments”.

However, each of these techniques present several disadvantages since tissue cuttings have not been reduced efficiently, therefore, post-operative pain is persistent, greater blood loss, longer hospital staying and longer recovery time; due to instrumentation as well as implants, which are still very sophisticated, and surgery is in a bilateral way or double wall.

Nevertheless, there are surgical methods of only one side, see, for example: North American Application No. 2005/0125065 A1 to Zucherman et al “Laterally insertable crossbar spacer”; North American Application No. 2005/0143820 A1 to Zucherman et al “Method of Laterally inserting an artificial vertebral disk replacement implant with translating pivot point”; International Application No. WO 00/44318 to Commarmond “Interbody vertebral implant with sagittal insertion”; International Application No. WO 2004/093691 to Liu et al “Methods and instrumentation for positioning implants in spinal disk space in an anterior lateral approach”; International Application No. WO 2006/104990 to Peterman et al “Spinal system and Method including lateral approach”.

However, each of these techniques present several disadvantages, since the implant above mentioned in the disclosed techniques in those documents is very inflexible and the instrumentation used in the corresponding surgery is too sophisticated, giving as a result the inconvenient above mentioned.

Regarding the stabilization interspinal devices, several of them are already known, implants which are placed are known as a reinforcement to the interspine ligament of the affected disk. One more example, which is known, is the DIAM® (Dynamic Intervertebral Assisted Motion or also known as Device for Intervertebral Assisted Motion), which was created by JEAN TAYLOR from the Clinique de L'Esperance in Nize. The surgery is carried out mainly in patients with disk degenerative illness or patients who present disk herniation.

The later dynamical enter spinal stabilization method through a balance device or DIAM® implant reduces the disk charge, restores tension of the later band aligning the line of the joint facet and increases the foraminal height. The DIAM® implant is butterfly-shaped (as an embodiment of presentation or shape), since it is the shape which adapts to such space, between the adjoining spinal apophysis of the harmed disk, whose core is made of silicone with external mesh and the tying is made of polyester. The device is available in a variety of sizes between 8 and 14 mm to allow the surgeon to place the most suitable to the patient's anatomy.

The method involves freeing tissues without harming ligaments in general as the upper ligament is not cut (supraligament) and through this site the surgeon will place the respective implant.

Since it is about a dynamic implant, it becomes a distraction force on the adjacent vertebras, detaching them so that the mechanical charge that falls on such structure decreases and enlarges the inter-vertebral foramen where nerves come out, which caused pain when being compressed.

Replacing the interspinal ligament diminishes pain, since it improves cushioning and reduces friction caused by the loss of volume or elasticity of the damaged disk.

The implant stabilizes the joint apophysis, therefore, it is a recommended method for patients with disk illness of degenerative kind. The operation is a relatively simple surgery not very invasive (the incision on the back skin is 5 or 6 cm long, approximately) and it does not require the surgeon intervention in the vertebral channel, therefore, the risk of neurological commitment is minimal, see the following bibliographical references:

a) Device for intervertebral assisted motion: technique and initial results, Neurosurg. Focus/Volume 22/January, 2007.

b) Posterior Dynamic Stabilization using the DIAM (Device for Intervertebral Assisted Motion). Jean Taylor. MD. Centre Hospitalier Princesse Grace—MONACO.

c) Classification of posterior dynamic stabilization devices, Department of Neurosurgery, University of Southern California, Los Angeles, Calif., Focus/Volume 22/January, 2007.

d) Patient Information Brochure, Medtronic Sofamor danek Australasia, 4/446 Victoria Road, Gladesville, NSW 2111.

e) Minimally Invasive Dynamic Stabilization of the Lumbar Motion Segment with an Intersinous Implant, Author: J. Sénégas, Minimally Invasive Spine Surgery; pp 459-465.

The techniques described in such documents also present several disadvantages, since they have not reduced tissues cuttings efficiently, persisting postoperative pain, blood loss, longer hospital staying and recovery time due to instrumentation which is still very sophisticated, and the surgery is in a bilateral way or double wall.

### SUMMARY OF THE DISCLOSURE

The present disclosure provides a save and single method, as well as a new instrumentation to improve the technique of minimally invasive spine surgery of only one side, where the new instrumentation relates to the use of special hooks and a new improved stabilization interspinal device which is placed for the reinforcement of the interspinal ligament of the affected disk, thus mitigating the pressure on spinal nerves.

An object of the present disclosure is to maintain the integrity of the spinal channel by carrying out an ambulatory surgery, with minimal cuttings in tissues and achieving, this way, the quick recovery and reduced costs in less time than the conventionally used for the same.

Another object of the present disclosure is minimizing pain, bleeding, hospital staying and achieving a better recovery of the patient as soon as possible.

Another object of the present disclosure is avoiding healing around spinal nerves by avoiding an exposure of double wall.

Another object of the present disclosure is providing a pair of hooks as new instrumental.

Another object of the present disclosure is providing an improved interspinal device of stabilization.

All the features and advantages above mentioned and others of the disclosure will be better understood through the following illustrative description, but not limiting the preferred embodiments regarding the attached drawings.

### BRIEF DESCRIPTION OF THE DRAWINGS

FIG. 1 shows a perspective view of a vertebra, freeing interspinal tissue through a curve Kerrison.

FIG. 2 shows a perspective view of a vertebra, where some threads are introduced to give amplitude to the interspinal space.

FIG. 3 shows a perspective view of a vertebra where a distractor is introduced and, this way, it measures the space with the purpose of selecting the size of the implant.

FIGS. 4a and 4b show a perspective view of a hook with angular ends to guide the sutures, according to the present disclosure.

FIG. 5 shows a perspective view of a new and improved interspinal device of stabilization or implant, according to the present disclosure.

FIGS. 6a to 6h show the essential steps of the sutures excursion, first and second, of the implant, according to the present disclosure.

FIG. 7 shows an implant in position to be placed between the respective apophysis.

FIGS. 8a to 8f show the positioning of an implant through a detractor, special clamps and a “holder”, respectively.

FIGS. 9a to 9c show the way of how to carry out sutures tying, according to the present disclosure.

FIGS. 10a to 10c show the way of how to carry out the fixed implant (DIAM®), according to the present disclosure.

### DETAILED DESCRIPTION

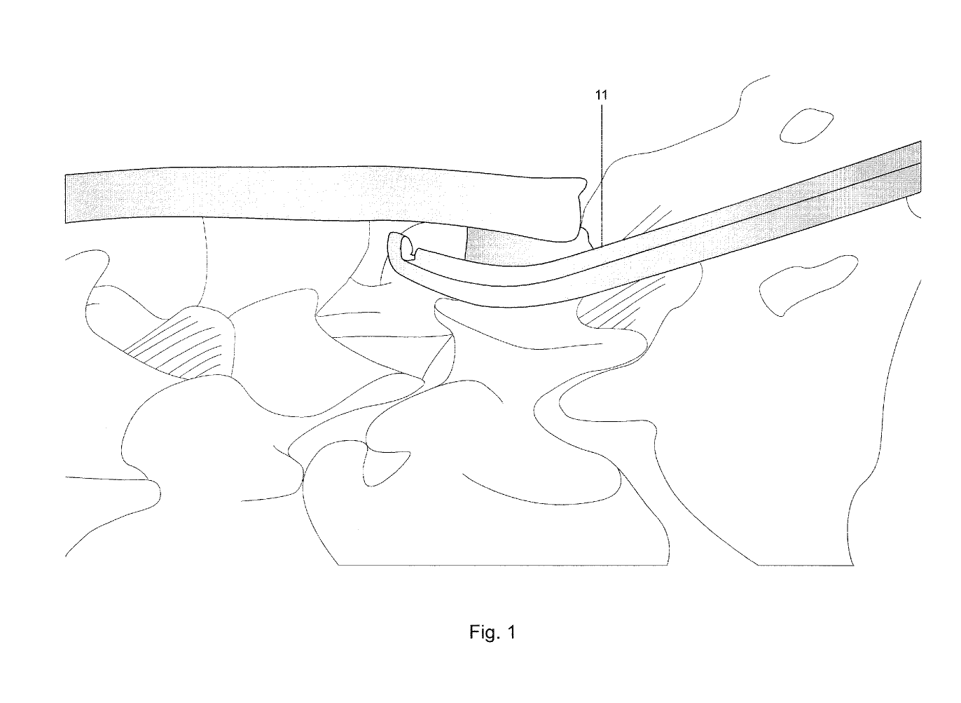

According to FIG. 1, a method to carry out a medial incision is introduced on a selected space where it is dissected and reflects (separates) the vertebral muscle without eliminating the supraspinal ligament by only leaving to sight the selected interspinal space, freeing, subsequently, the former part of the interspinal ligament through a curve Kerrison clamp (11); however, respecting the rear part and, thus, make the excursion of the suture easier.

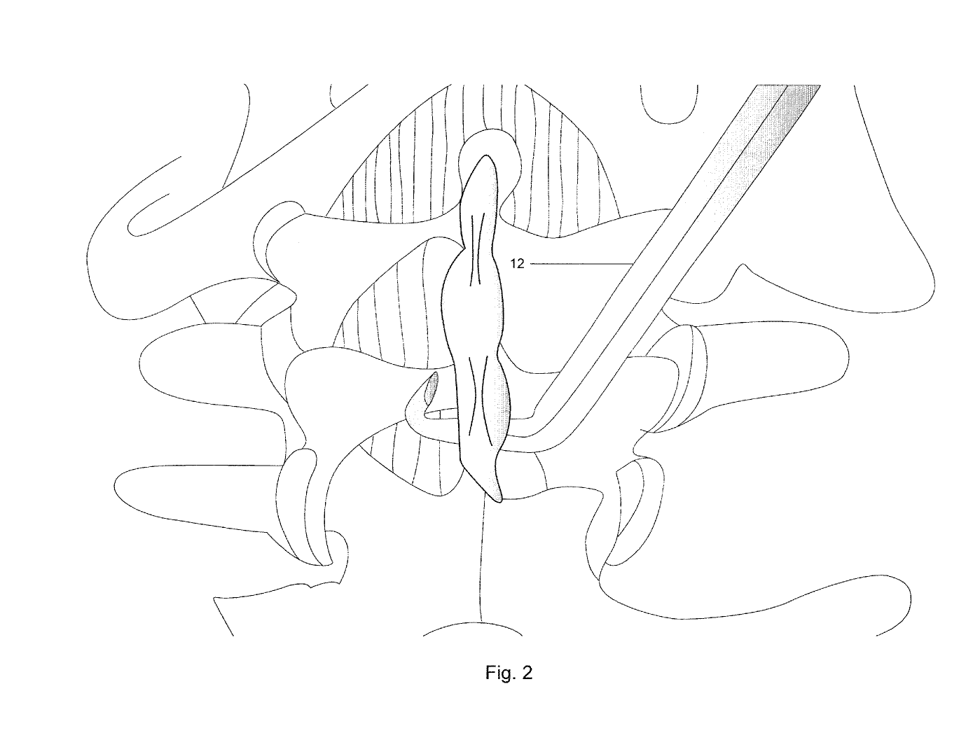

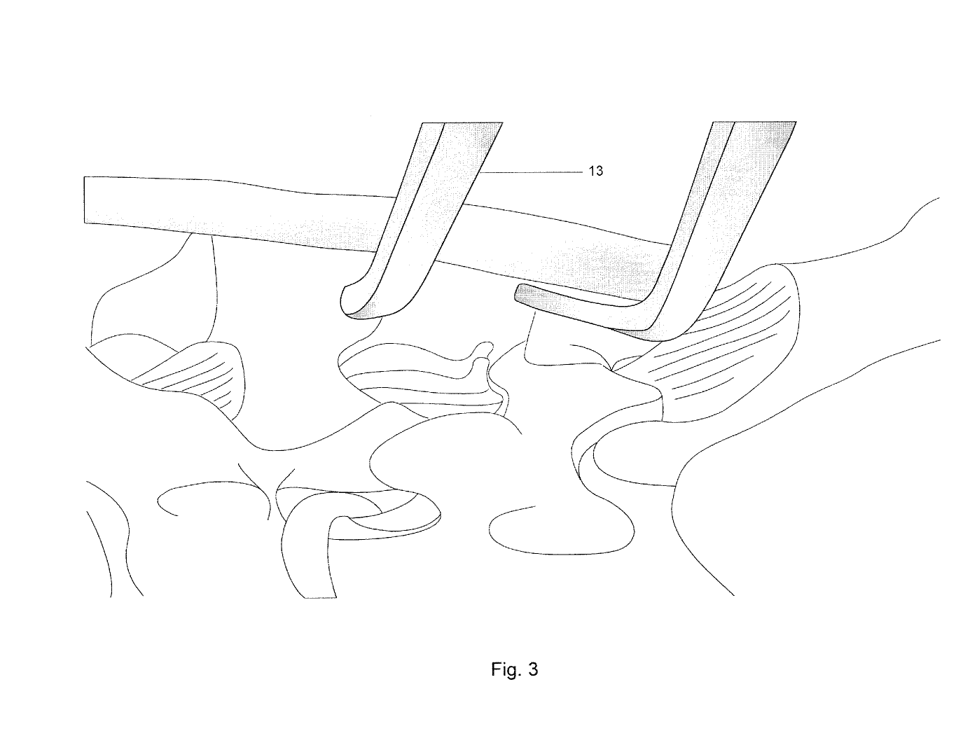

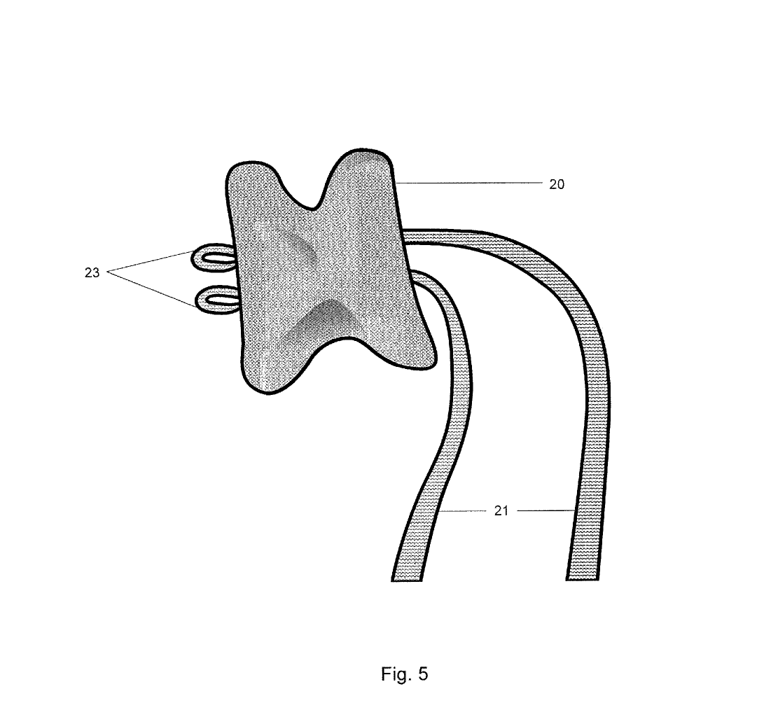

Subsequently and according to FIG. 2, some chisel are introduced (12) to give amplitude in the space that the anchoring suture will go through (see FIG. 5). With the object of performing the distraction of the space, a distractor is introduced (13); see FIG. 3 and, this way, measure the space with the purpose of selecting the size of the implant.

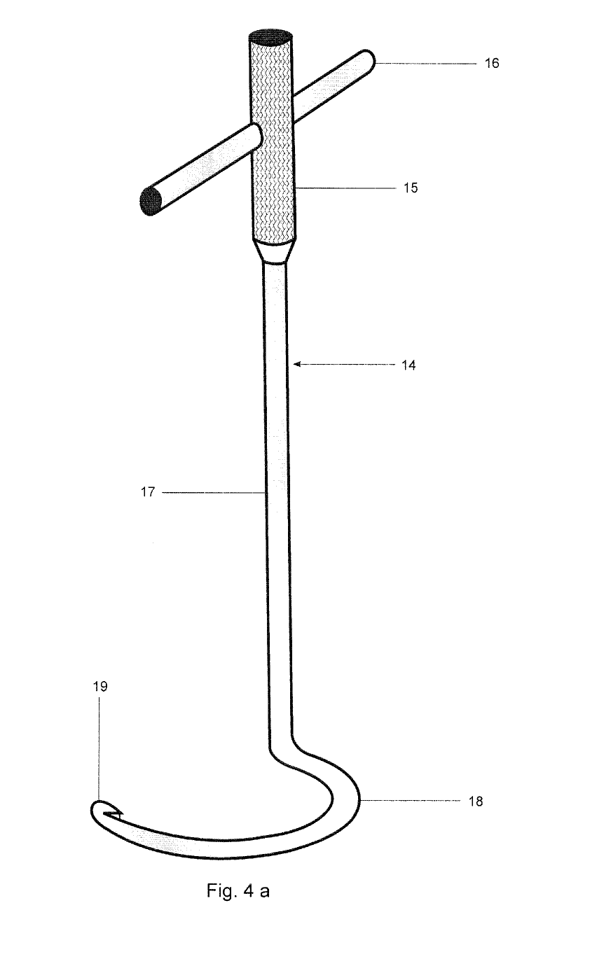

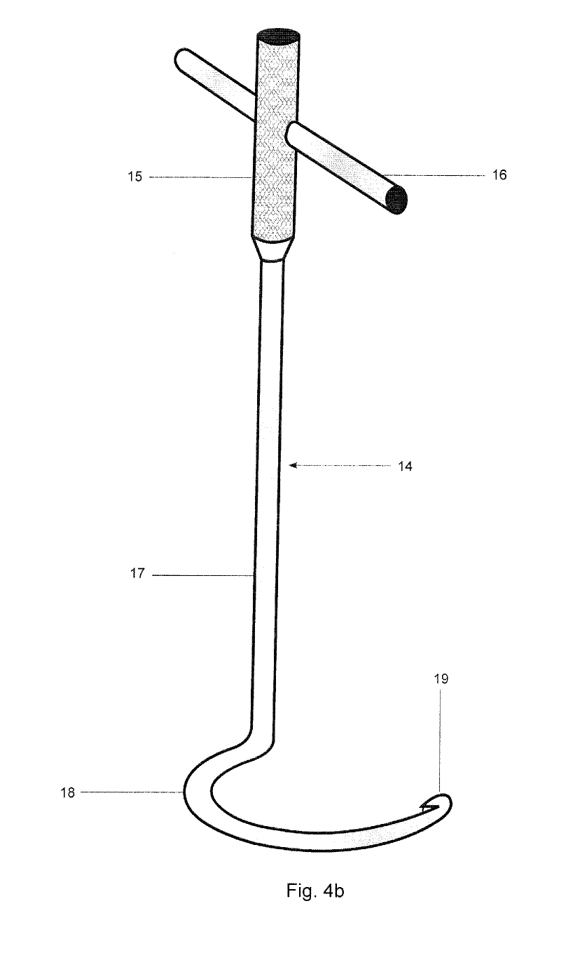

FIGS. 4a and 4b show a perspective view of a pair of hooks (14) with angular ends, left and right respectively, to guide the suture around the spinal apophysis base. Each hook (14) is made of the group comprising stainless steel and/or dark matched titanium; this is efficiently advisable since it avoids surgeon dazzling by light reflection, and with a length of 21 cm.

Each hook presents a cylindrical body (15), whose external surface presents a knurl for a better adhesion to the retention, where in one end of it a retention sleeve is disposed fixedly and transversally (16), and on the other end of the cylindrical body (15) an enlarged rod is bonded fixedly (17), which terminates with a curve end (18) placed at 90 degrees regarding the axis of the enlarged rod (17) and with a fishhook-shaped tip (19), where such curve end (18) presents a curvature continuity of 60±15 degrees and a length of arch of 50±10 mm.

According to FIG. 5, the new implant (20) basically has the shape of a butterfly to adapt to the shape between the two spinal apophysis adjoining the damaged disk. Such implant (20) is made of silicone with external mesh and the sutures (21) are made of polyester. The implant is available in a variety of sizes from 8 to 14 mm.

The improved implant (20) presents, in its frontal part, two adjoining buttonholes (23) and laterally parallel among them, and in the rear part the pair of sutures are disposed (21) parallely disposed corresponding among them.

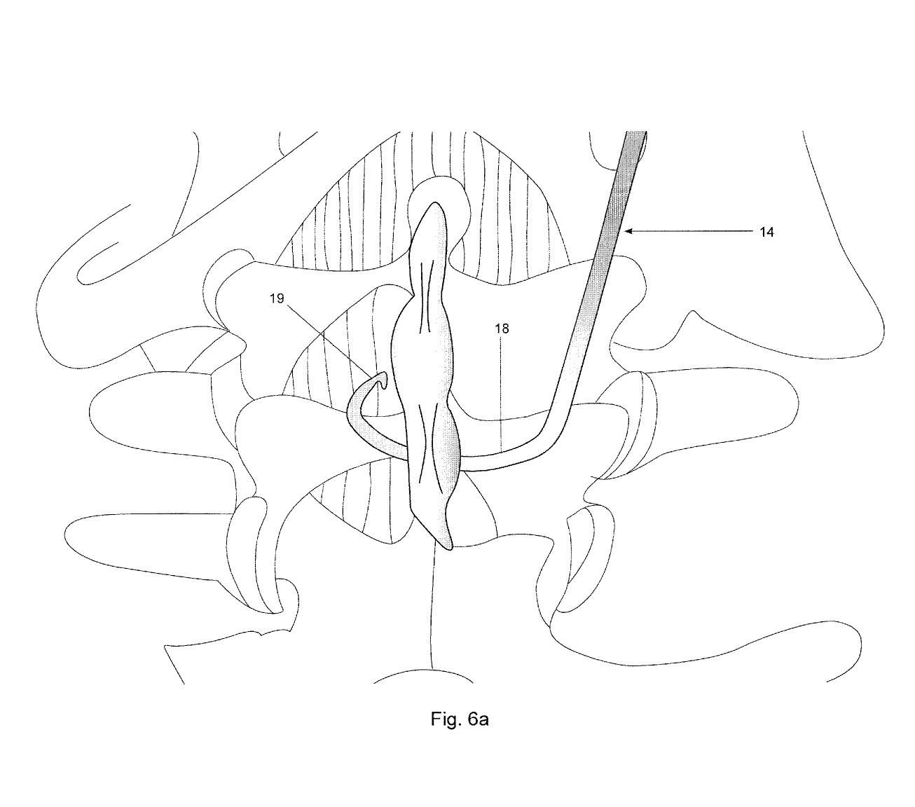

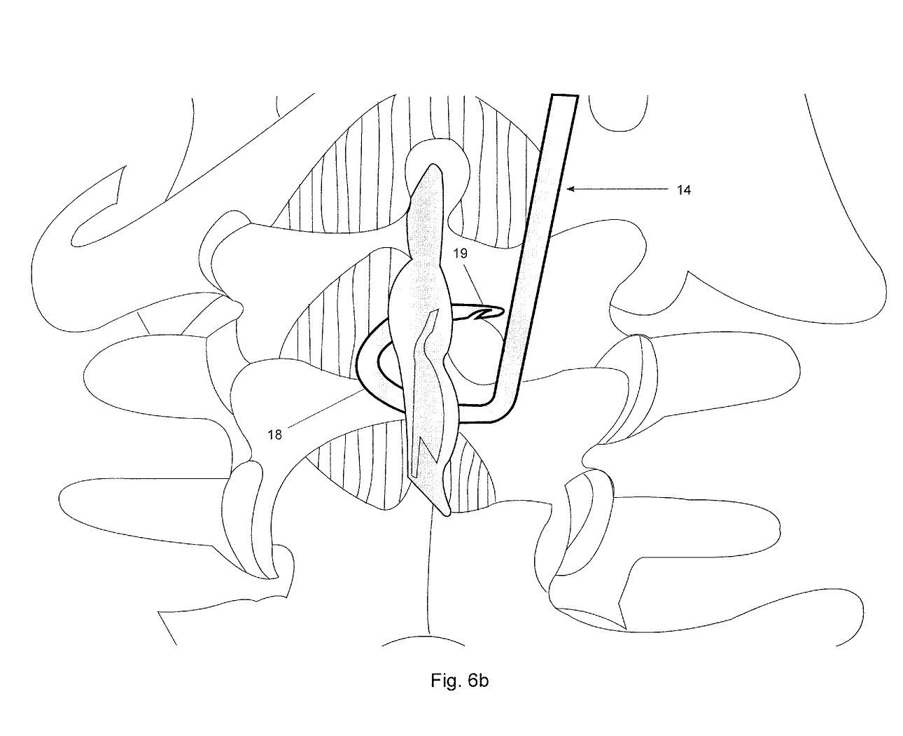

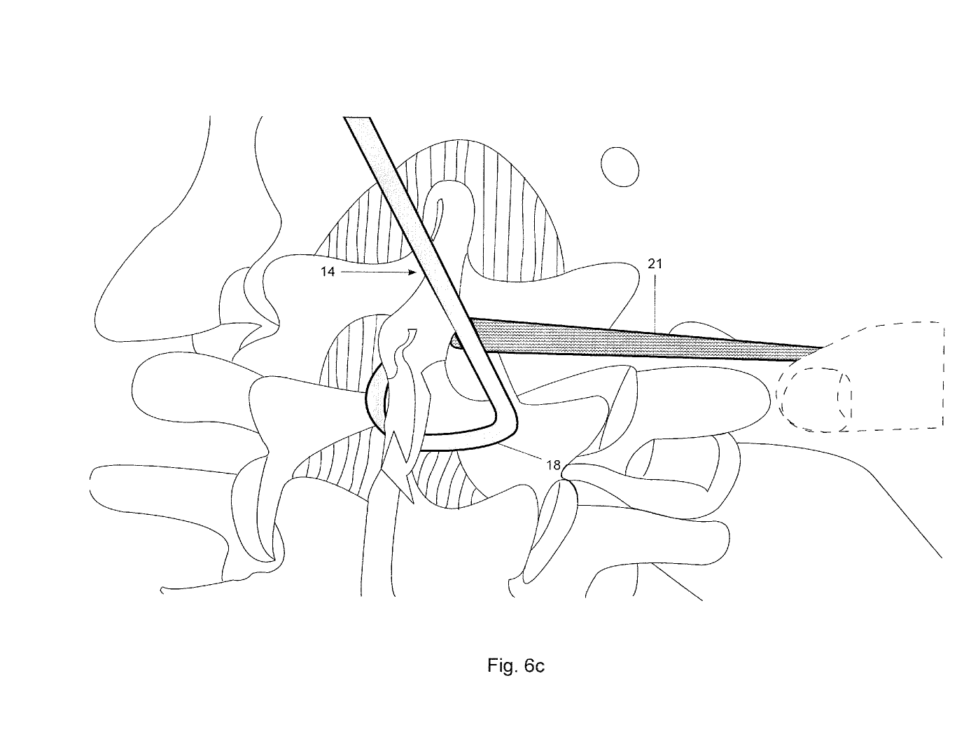

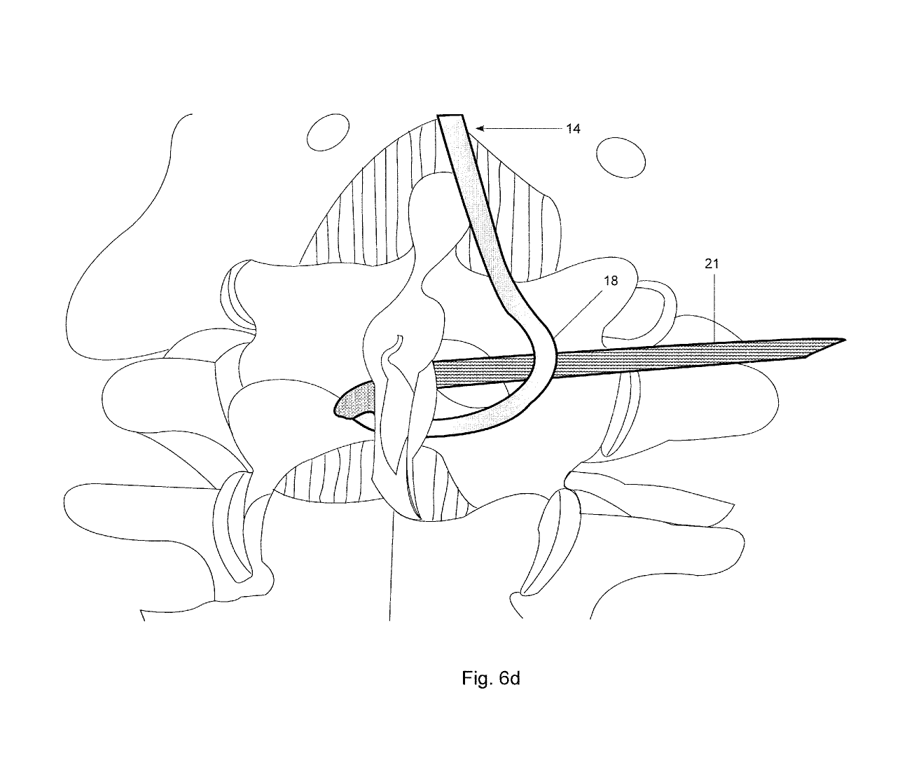

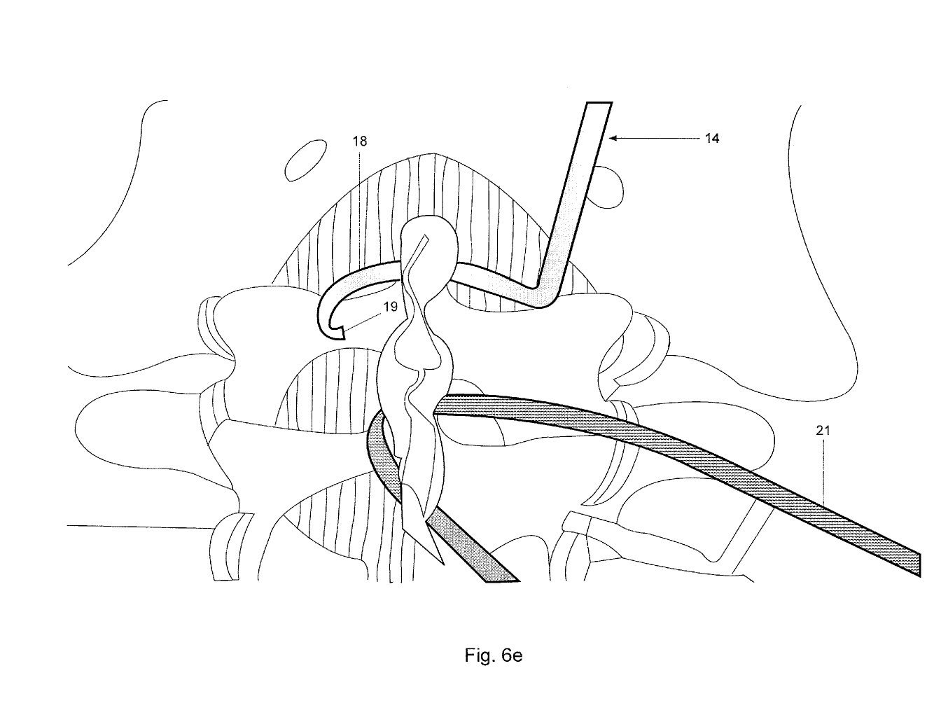

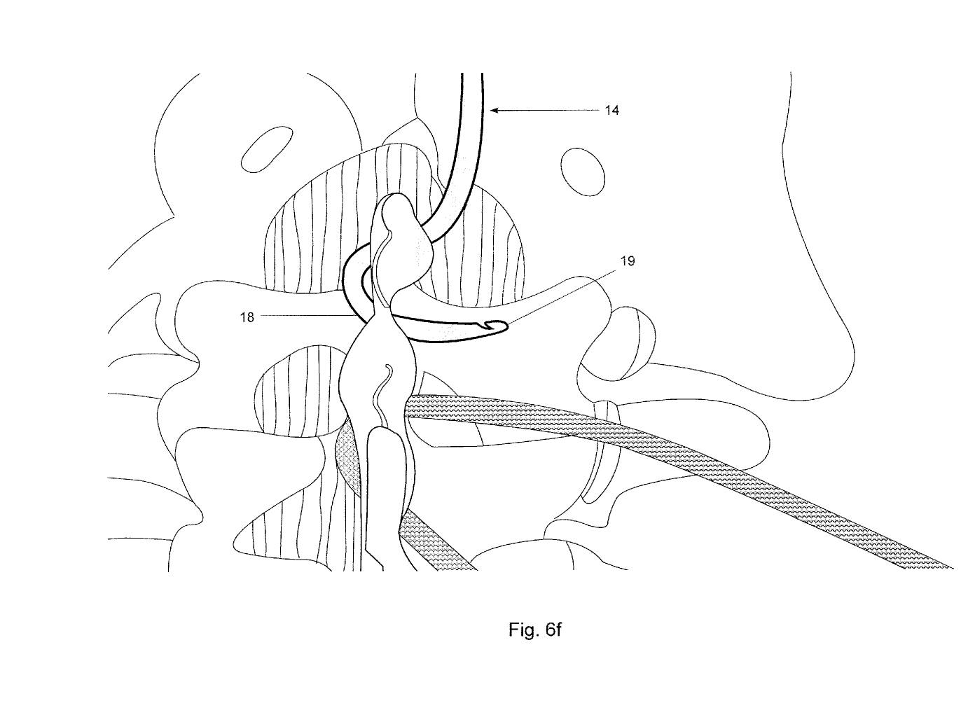

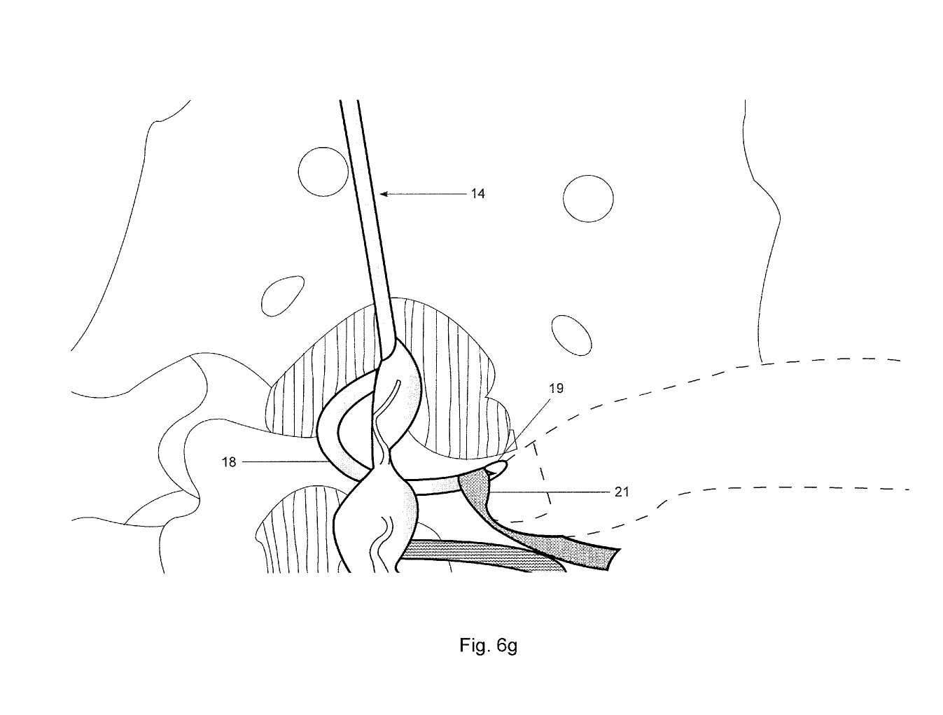

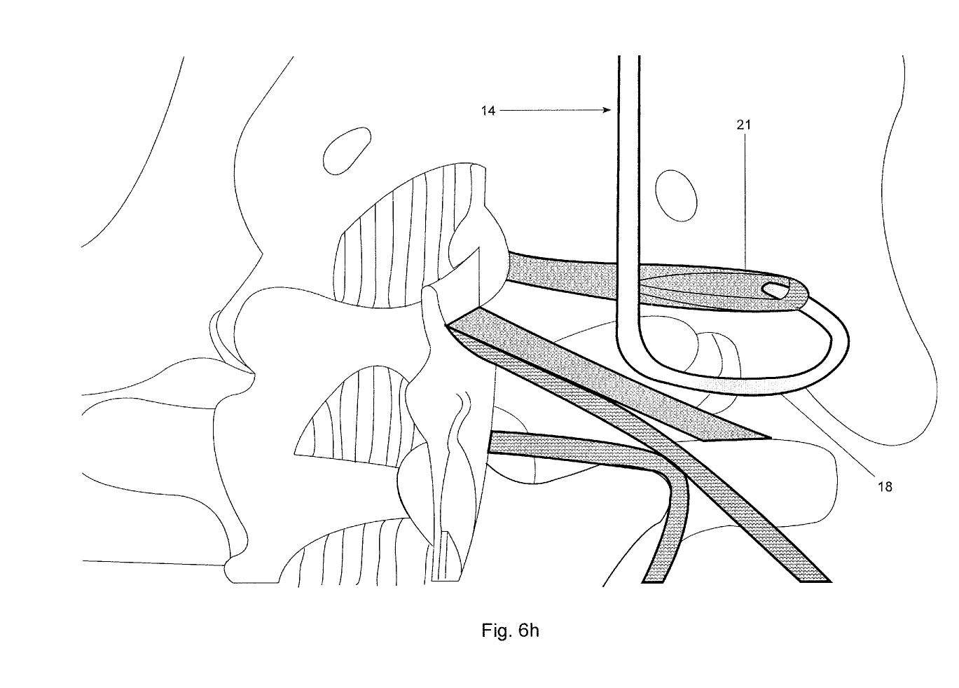

In FIGS. 6a to 6h, the essential steps are seen for the positioning of the implant (DIAM®), where it is important to mention first, in an illustrative and explanatory way, the identification of the first apophysis and the second apophysis, where the first apophysis is the closest to the head and the second apophysis being the closest to the sacrum region. FIGS. 6a and 6b respectively, illustrate the left hook (14), which the curve end (18) with fishhook-shaped tip (19) is rotated, at the back and front regarding the first apophysis. Once this step is carried out, the suture (21) is then hooked with the fishhook-shaped tip (19) and the suture (21) is introduced by one side of the base of the first apophysis by rotating in the opposite direction of the hook (14), see FIGS. 6c and 6d. Similarly, the same steps are performed to make the second suture (21) pass around the second apophysis with the right hook (14), see FIG. 6e to 6h.

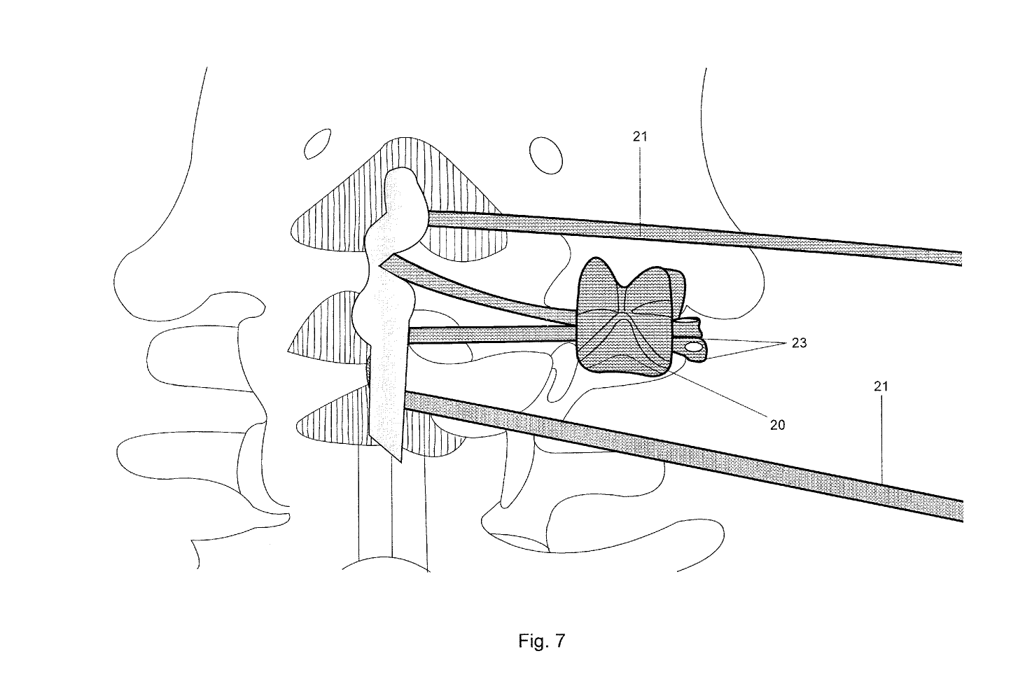

Once the introduction of sutures (21) is carried out in the first and second apophysis, the implant (20) remains in position as is clearly seen in FIG. 7.

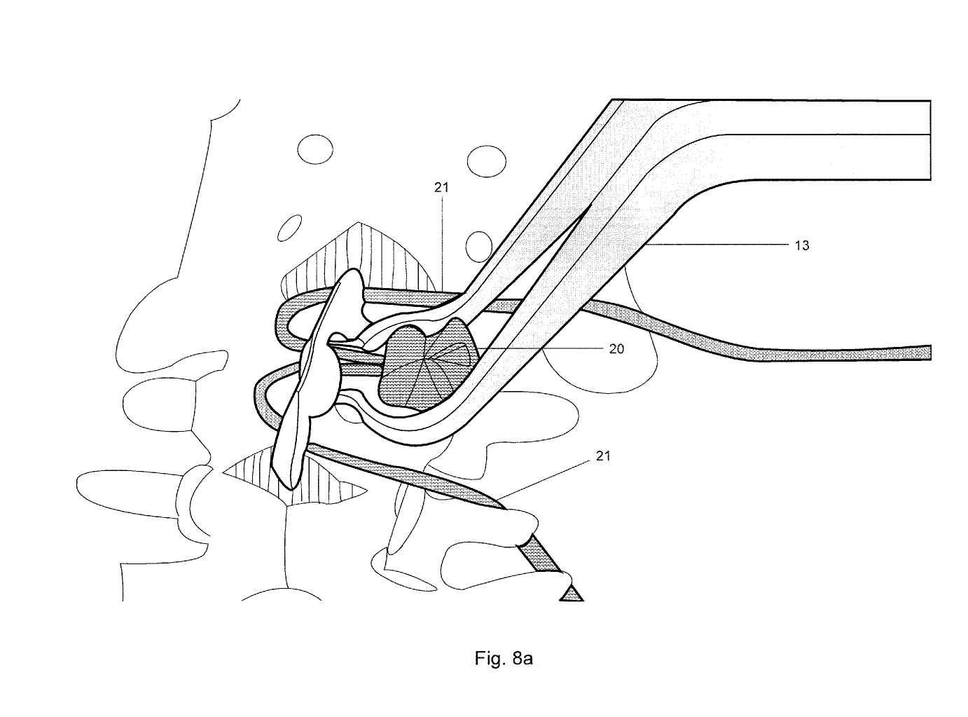

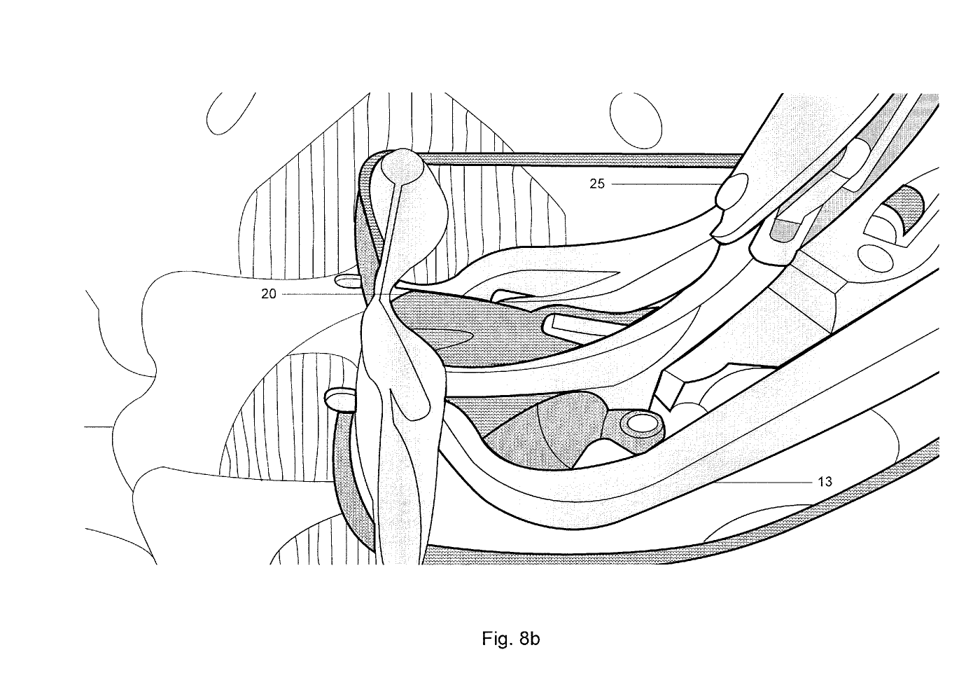

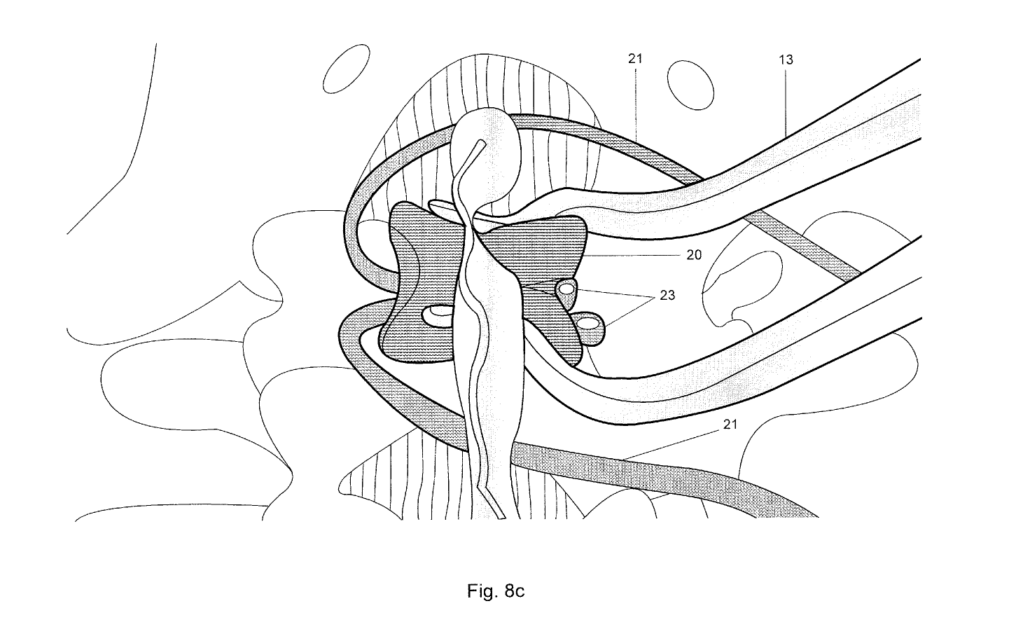

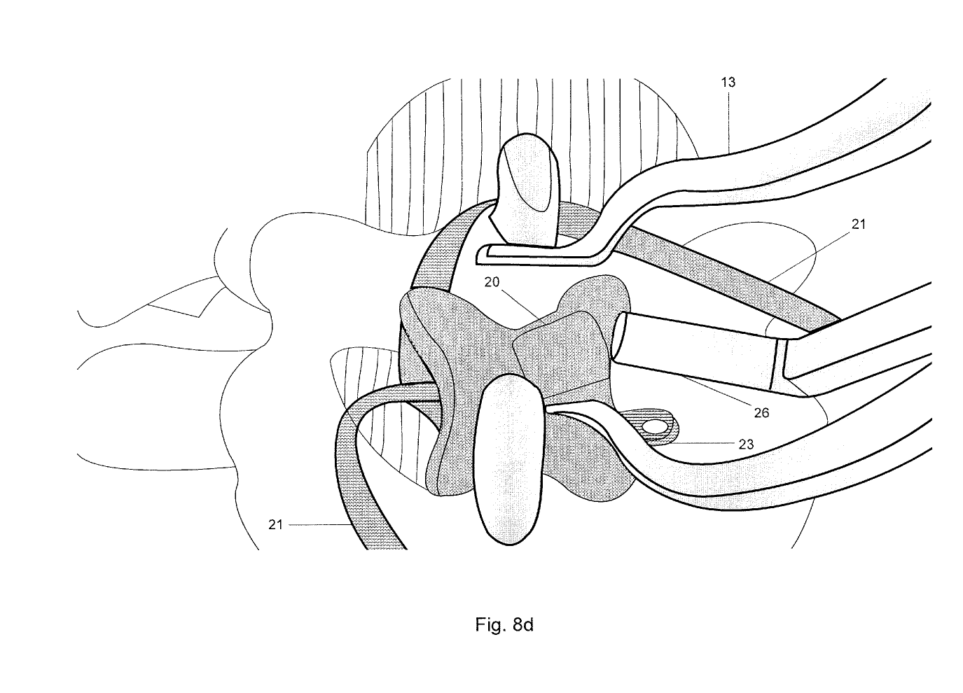

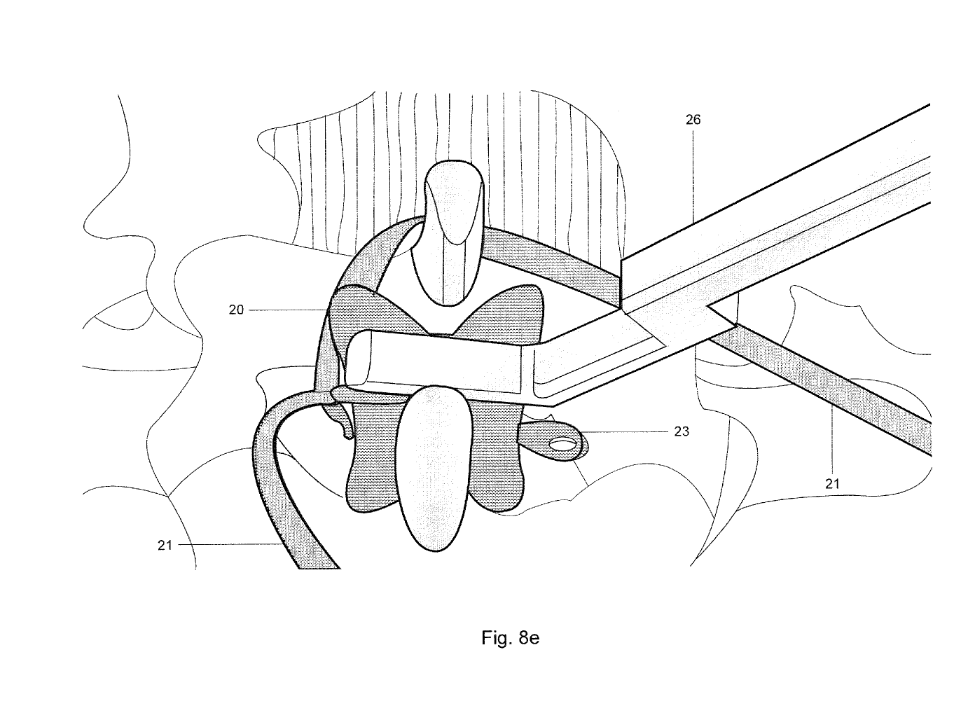

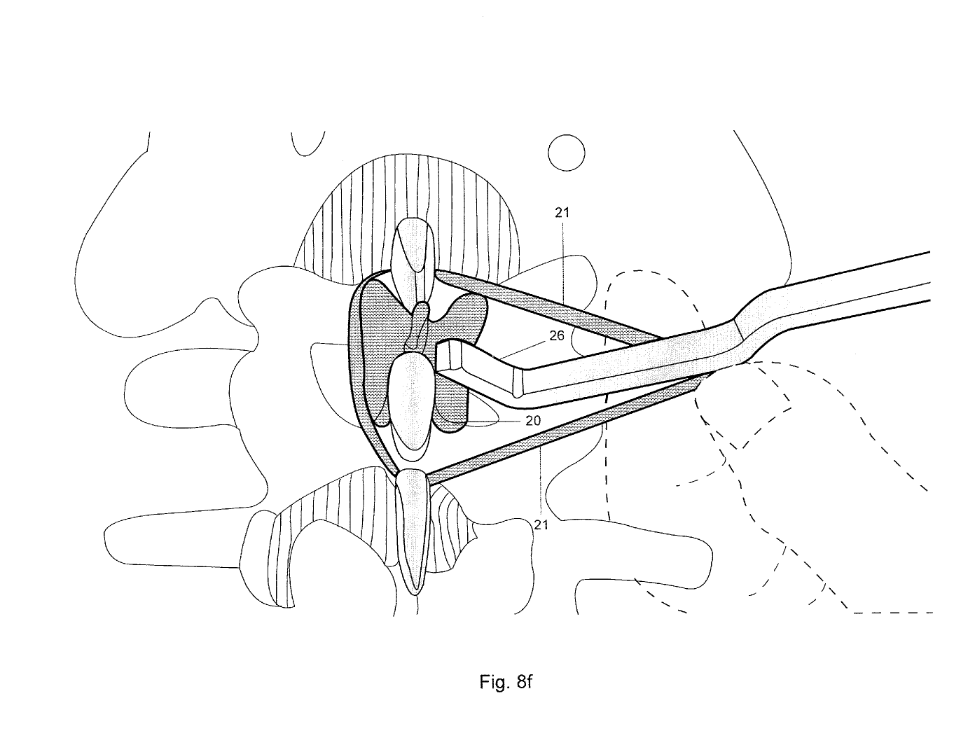

Subsequently, the distractor (13) is placed again to open the interspinal space and give enough space for the positioning of the implant, see FIG. 8a, then the implant is fastened through the special clamp (25), see FIG. 8b, the implant is introduced and placed between the first and second apophysis, see FIG. 8c. Once these steps are performed, the distractor (13) is taken away and the implant (20) is placed through a “holder” (26) and, this way, the implant is placed perfectly well between the first and second apophysis, see FIGS. 8d to 8f (in these figures, the part which represents the supraspinal ligament has been eliminated with the purpose of clearly visualizing the implant positioning).

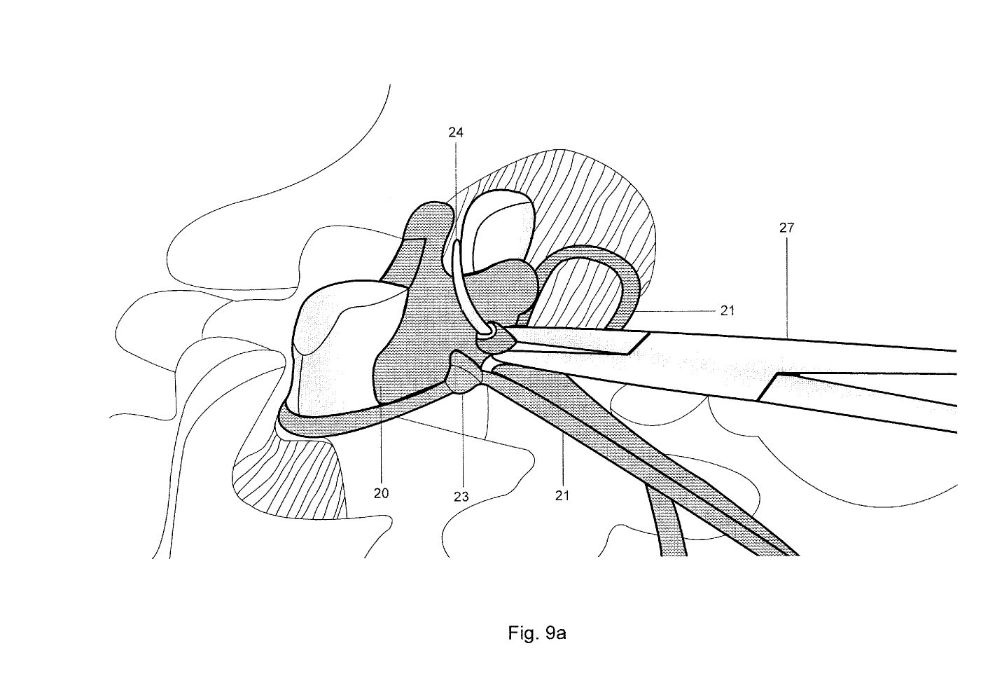

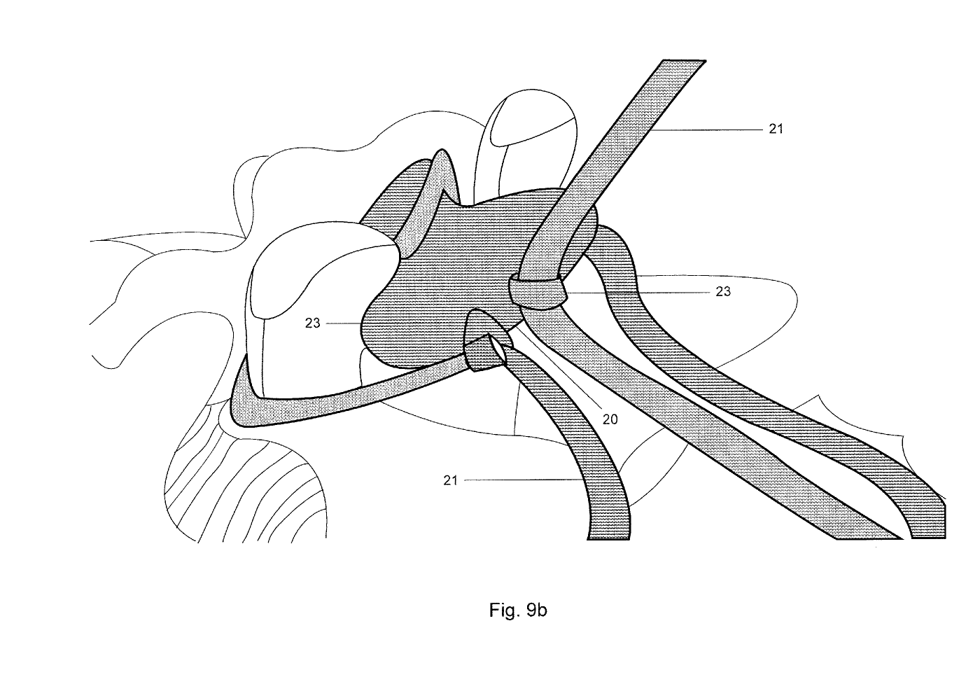

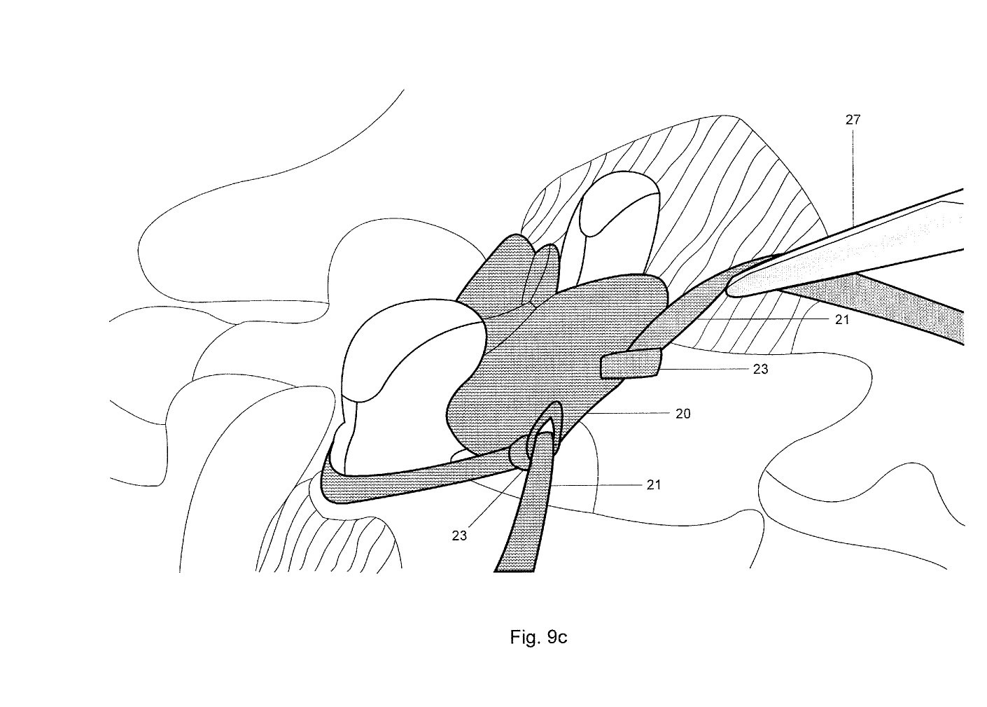

In FIGS. 9a to 9c, the anchoring of both sutures (21) is observed through the frontal side of the implant (20); for this, a needle (24) is passed through with the help of a sharp clamps (27) and which is joined to the free end of each suture (21), through the buttonholes (23), and both sutures (21) are pulled to improve the positioning of such implant (20) (once again, in these Figures the part which represents the supraspinal ligament has been eliminated with the purpose of visualizing the implant positioning).

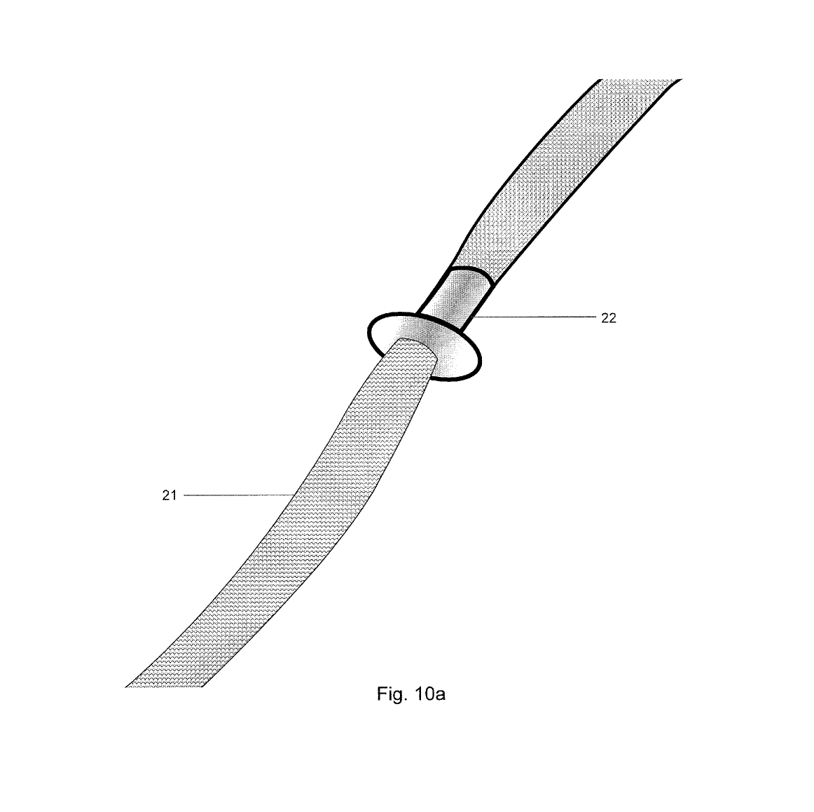

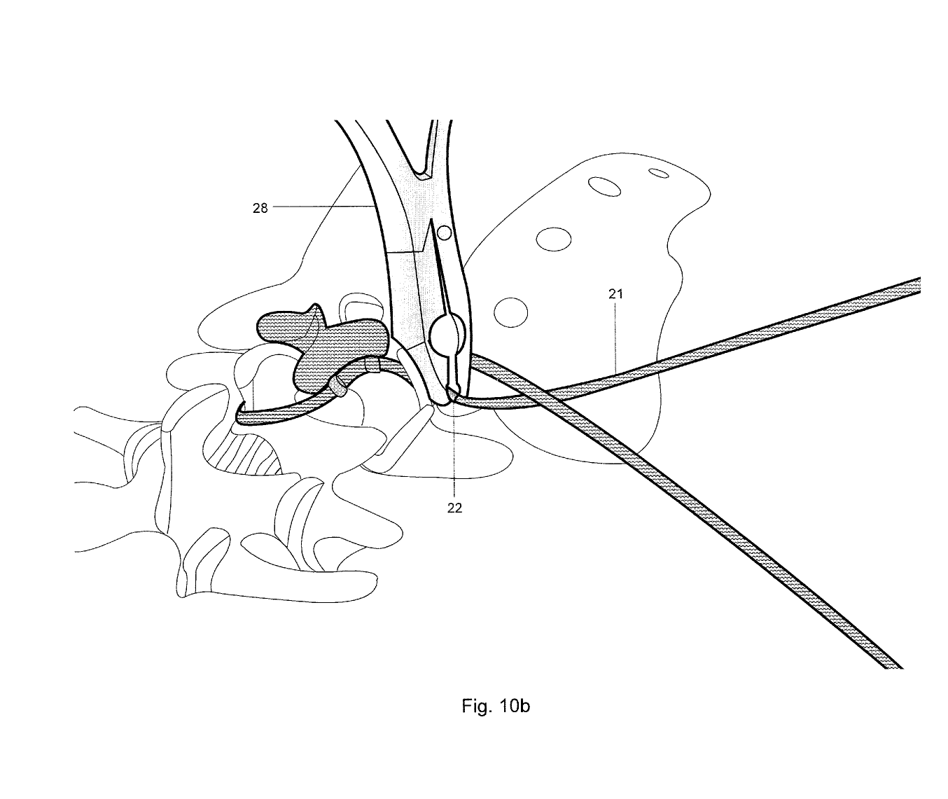

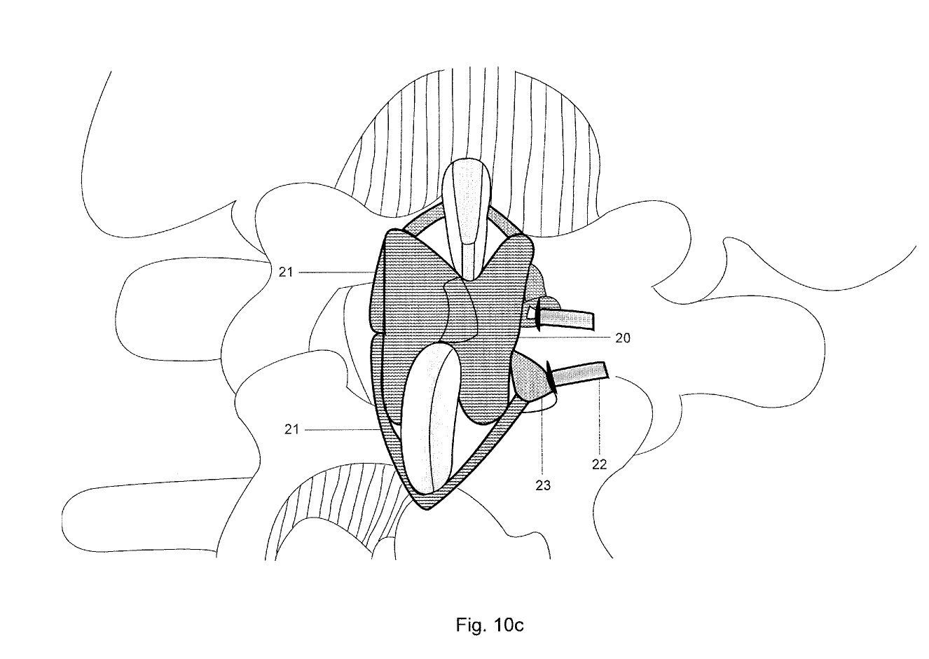

Once transferred, each suture (21) is placed in a slipping way through a strapping clamp (28), a retention bushing (22) made of titanium to the frontal side of the implant (20) and through this strapping clamp (28) the bushing walls are pressed in such a way that said bushing (22) is fastened to the respective suture, getting rid at the same time of the suture residues (21), see FIGS. 10a and 10c (once again, in these Figures the part which represents the supraspinal ligament has been eliminated with the purpose of clearly visualizing the implant positioning). Finally, the deep layers and skin are closed.

Now then, since the fifth lumbar vertebra (L5) is jointed to the sacrum (five vertebras merged S1 -S5), an anchoring may be performed in L5-S1 without including an anchoring screw in S1, which greatly simplifies the procedures and risks of operation.

If the patient presents spinal apophysis in S1, the technique is carried out the same way above mentioned.

In a particular case, if there is not spinal apophysis in S1, it is necessary to carry out an additional step before the use of hooks, which includes the use of an osteotome to perform an osteotomy or slot in the base of the spinal apophysis of S1 which may be from 1 to 4 mm depth to allow the suture to be fastened in such slot and the procedure continues the same stages previously disclosed.

### Statistics

Statistics show a complication rate lower than 3%, the main reason being fracture of spinal apophysis. Nevertheless, one of its great advantages is that it always gives the option of another procedure in case it is required. However, there are some contradictions since the implant DIAM® is recommended, mainly, in patients with disk degenerative illness, i.e., whose disk has suffered wearing away or, in some cases, disk herniation. The DIAM® implants are not provided deviation of spine (scoliosis). For a better understanding, in the following example some satisfying features on the use of DIAM® implant may be observed:

### EXAMPLE

50 patients with hernia of one or two disks:

| | |
|---|---|
| Woman | 13 (43.8 years old average) |
| Man | 37 (43.4 years old average) |

Levels:

| | |
|---|---|
| L2-L3 | 1 |
| L3-L4 | 1 (Wallis L4-L5) |
| L4-L5 and L5-S1 | 4 |
| L4-L5 | 34 |

Results:

| | |
|---|---|
| Excellent | 48 patients |
| Good | 1 (overweight) |
| Regular | 1 (Osteoporosis) |

Times:

| | |
|---|---|
| Surgery (one side) | 1 level 40 minutes |
| | 2 levels 60 minutes |

Hospitalization:
8 hours (39 patients, i.e. 78%)
1 day (8 patients, i.e. 16%)
2 days (3 patients, i.e. 6%)

Pain after surgery (1-3 days):

| | |
|---|---|
| Light | 35 |
| Medium | 8 |
| High | 7 |

| | | |
|---|---|---|
| Back to work | 10 days | 40 |
| | 15 days | 3 |
| | Retired | 7 |

After 3 months:
46 patients (excellent results without pain)
2 patients (good results, light pain)
2 patients abandoned the treatment.

After 9 months:
45 patients (remain excellent)
2 patients (remain with good results)
3 patients abandoned the treatment.

After 18 months:
45 patients (remain excellent)
2 patients (remain with good results)
3 patients abandoned the treatment.

Obviously, several modifications and variations to the disclosure may be performed as set forth in the present disclosure without departing from the scope and spirit of it and, therefore, these limitations will only be imposed as indicated by the attached claims.

1. A pair of hooks for its use in a method for surgery of minimally invasive spine in interspinal space of only one side, comprising:
a cylindrical body;
a retention sleeve placed in a tranversally and fixed to the upper part of the cylindrical body;
an enlarged rod placed fixedly in the lower part of the cylindrical body with a curve end and placed at 90 degrees regarding the axis of the enlarged rod.

2. The hook of claim 1, wherein the curve end of the first hook is bent to the left.

3. The hook of claim 1, wherein the curve end of the second hook is bent to the right.

4. The hook of claim 1, wherein the curve end of each hook presents a fishhook-shaped tip.

5. The hook of claim 1, wherein an external surface of the cylindrical body of each hook presents a knurled surface for a better adhesion to retention.

6. The hook of claim 1, wherein the curve end of each hook presents a curvature continuity of 60±15 degrees and an arch length of 50±10 mm.

7. The hook of claim 1, wherein each hook is made of the group comprising stainless steel and/or dark matched titanium.

8. The hook of claim 1, wherein each hook presents a total length of about 21 cm.

9. An improved implant for the replacement of the interspinal ligament and for its use in a method for spine surgery in minimally invasive interspinal space of only one side, comprising:
a butterfly-shaped body basically made of silicone and covered with an external mesh made of polyester to adapt to the form between the two spinal apophysis adjacent to the damaged disk;
a pair of sutures made of polyester.
a pair of buttonholes; and
a retention bushing is placed in each suture;
wherein, on a frontal side of such implant there are two adjacent buttonholes and laterally parallel among them; and
on a rear side of such implant there are two adjacent sutures and laterally parallel among them.

10. The implant of claim 9, wherein the size of such implant is about 8 to 14 mm.

11. The implant of claim 9, wherein the retention bushing is made of titanium.

12. A method for a spine surgery in invasive minimal interspinal space of only one side, comprising:
carrying out a medial incision on the space selected where is dissected and reflects (separates) the vertebral muscle leaving to sight the selected interspinal space;
removing the former part of the interspinal ligament through a curve Kerrison, but respecting the rear part and thus making the excursion of the suture easier;
introducing a chisel to give amplitude in the space that the anchoring suture of the implant will go through;
introducing a distractor to measure the space without exaggerating the distraction space;
introducing in a rotatory way a first hook to guide the first suture of the implant surrounding the spinal apophysis base;
introducing in a rotatory way a second hook to guide the second suture of the implant surrounding the other spinal apophysis base;
placing the distractor clamps once again to open the interspinal space;
placing the implant between the spinal apophysis through a special clamp;
removing the distractor clamps and place the implant through a guide clamp to place the implant perfectly between the first and second apophysis;
anchoring both sutures on the frontal side of the implant passing a needle through, which is joined in the free end of each suture, through the buttonholes with the aid of a clamp, and pull both sutures to improve the positioning of such implant;
placing in a slipping way, through a clamp, a retention bushing to the frontal wall and pressing the walls of the bushing in such a way that said bushing is fastened to the respective suture; and
closing the deep layers and the skin.

13. The method of claim 12, wherein both hooks, left and right, circulate at the back and front regarding the first and second apophysis.

14. The method of claim 12, wherein the additional step of carrying out an osteotomy or slot on the base of the spinal apophysis of S1 from 1 to 4 mm depth is performed to allow the suture to fasten in that slot, if there is not spinal apophysis in S1.
